(19) United States
(12) Patent Application Publication
(10) Pub. No.: US 2024/0398683 A1
(43) Pub. Date: Dec. 5, 2024

(54) WATER-IN-OIL EMULSION COSMETIC

(71) Applicant: SHISEIDO COMPANY, LTD., Tokyo (JP)

(72) Inventor: Keita NISHIDA, Tokyo (JP)

(73) Assignee: SHISEIDO COMPANY, LTD., Tokyo (JP)

(21) Appl. No.: 18/699,486

(22) PCT Filed: Oct. 26, 2022
(86) PCT No.: PCT/JP2022/039833

§ 371 Date: Apr. 8, 2024

(30) Foreign Application Priority Data
Nov. 8, 2021 (JP) 2021-181993

(51) Int. Cl. A61K 8/49; A61K 8/06; A61K 8/46; A61Q 19/00

(52) U.S. Cl. CPC A61K 8/4946; A61K 8/064; A61K 8/463; A61K 8/466; A61K 8/4913; A61Q 19/00; A61K 2800/10

(57) ABSTRACT

[Problem] To provide a water-in-oil emulsion cosmetic having an excellent feel in use.

[Solution] A water-in-oil emulsion cosmetic including (A) a cyclic carboxamide derivative having a specific structure or its salt, (B) a water-soluble benzylidene camphor derivative or a benzylidene camphor derivative salt, (C) an oil, and (D) water.

### TECHNICAL FIELD

The present invention relates to a water-in-oil emulsion cosmetic.

### BACKGROUND ART

Ultraviolet absorbers to be blended in sunscreen cosmetics include oil-soluble ultraviolet absorbers and water-soluble ultraviolet absorbers, and an oil-soluble ultraviolet absorber is often blended from the viewpoints of ultraviolet absorbing power, ease of handling, cost, and the like. However, an oil-soluble ultraviolet absorber causes a sticky feel. Therefore, blending a water-soluble ultraviolet absorber has also been studied, and for example, a water-in-oil emulsion cosmetic including a water-soluble ultraviolet absorber having a sulfonate group has been proposed (Patent Literature 1). The users of such a cosmetic have desired a more excellent feel in use, and there has been room for further improvement in the feel in use.

Meanwhile, a cyclic carboxamide derivative is known to have an anti-wrinkle effect and a pigmentation inhibitory effect, and is proposed to be blended in a cosmetic and the like (Patent Literature 2).

### CITATION LIST

### Patent Literature

Patent Literature 1: JP 2007-217393 A
Patent Literature 2: WO 2011/040496 A

### SUMMARY OF THE INVENTION

In water-in-oil formulation, there is room for improvement in a feel in use such as an oily feel. The present inventors have surprisingly found that an excellent feel in use can be achieved by using a water-in-oil cosmetic including a cyclic carboxamide derivative and a water-soluble benzylidene camphor derivative or a benzylidene camphor derivative salt. The present invention is based on these findings.

According to the present invention, the following invention is provided.

[1] A water-in-oil emulsion cosmetic including:
(A) a cyclic carboxamide derivative or a salt of the cyclic carboxamide derivative, the cyclic carboxamide derivative represented by Formula (1):

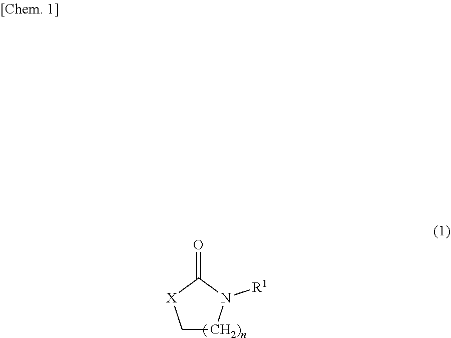

wherein
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3;
(B) a water-soluble benzylidene camphor derivative or a benzylidene camphor derivative salt;
(C) an oil; and
(D) water.
   [2] The cosmetic according to [1],
wherein in Formula (1) of the component (A),
R¹ represents a hydroxyalkyl group having 1 to 3 carbon atoms,
X represents —CH₂— or —NH—, and
n is 1.
   [3] The cosmetic according to [1] or [2], wherein the component (A) is 1-(2-hydroxyethyl)-2-imidazolidinone.
   [4] The cosmetic according to any one of [1] to [3], wherein an amount of the component (A) blended is 0.05 to 5 mass % with respect to a total amount of the cosmetic.
   [5] The cosmetic according to any one of [1] to [4], wherein the component (B) is selected from the group consisting of terephthalylidene dicamphor sulfonic acid, benzylidene camphor sulfonic acid, and camphor benzalkonium methosulfate.
   [6] The cosmetic according to any one of [1] to [5], wherein an amount of the component (B) blended is 0.05 to 10 mass % with respect to the total amount of the cosmetic.

According to the present invention, a water-in-oil emulsion cosmetic having an excellent feel in use can be provided. In particular, smoothness and freshness can be imparted without causing an oily feel during application.

### BEST MODE FOR CARRYING OUT THE INVENTION

The present invention relates to a water-in-oil emulsion cosmetic (hereinafter, sometimes referred to as a cosmetic) including (A) a cyclic carboxamide derivative having a specific structure or its salt, (B) a water-soluble benzylidene camphor derivative or a benzylidene camphor derivative salt, (C) an oil, and (D) water.

### (A) Cyclic Carboxamide Derivative or its Salt

The cosmetic according to the present invention includes (A) a cyclic carboxamide derivative represented by Formula (1) or its salt (hereinafter, sometimes referred to as a component (A), and the same applies to other components).

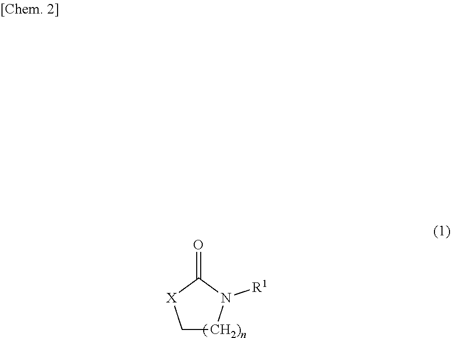

In Formula (1),
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3

The hydrocarbon group is not particularly limited, and may be, for example, an alkyl group, a cycloalkyl group, an alkenyl group, an alkynyl group, a cycloalkylalkyl group, a haloalkyl group, an alkoxyalkyl group, or an alkoxycarbonylalkyl group, and is preferably an alkyl group.

In a preferred embodiment, in Formula (1) of the component (A),
R¹ represents a hydroxyalkyl group having 1 to 3 carbon atoms,
X represents —CH₂— or —NH—, and
n is 1.

Specific examples of the cyclic carboxamide derivative represented by Formula (1) include the following.

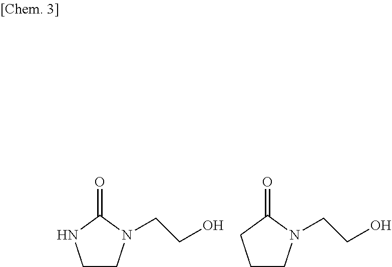

The component (A) is most preferably 1-(2-hydroxyethyl)-2-imidazolidinone.

The component (A) may be a salt of the cyclic carboxamide derivative represented by Formula (1). The kind of the salt is not particularly limited as long as the salt is pharmacologically acceptable, and the salt may be an inorganic salt or an organic salt. Examples of the inorganic salt include a hydrochloride, a sulfate, a phosphate, a hydrobromide, a sodium salt, a potassium salt, a magnesium salt, a calcium salt, a magnesium salt, and an ammonium salt. Examples of the organic salt include an acetate, a lactate, a maleate, a fumarate, a tartrate, a methanesulfonate, a p-toluenesulfonate, a triethanolamine salt, and an amino acid salt.

One or more kinds of the component (A) can be blended. The amount of the component (A) blended is preferably 0.05 to 5 mass %, and more preferably 0.5 to 3.5 mass % with respect to the total amount of the cosmetic.

### (B) Water-Soluble Benzylidene Camphor Derivative or Benzylidene Camphor Derivative Salt

The cosmetic according to the present invention includes (B) a water-soluble benzylidene camphor derivative or a benzylidene camphor derivative salt. The component (B) is a water-soluble ultraviolet absorber, and is preferably present in the aqueous phase. Although benzylidene camphor generally has low water solubility, the component (B) used in the present invention has a benzylidene camphor skeleton and simultaneously has a water-soluble group and can be stably present in the aqueous phase.

In the case of the component (B) being a water-soluble benzylidene camphor derivative, the component (B) preferably has a sulfo group. In the case of the component (B) being a benzylidene camphor derivative salt, the kind of the salt is not particularly limited as long as the salt is a pharmacologically acceptable salt, and the salt may be an inorganic salt or an organic salt and is preferably a sulfonate salt.

The component (B) is more preferably selected from the group consisting of terephthalylidene dicamphor sulfonic acid, benzylidene camphor sulfonic acid, and camphor benzalkonium methosulfate.

One or more kinds of the component (B) can be blended. The amount of the component (B) blended is preferably 0.05 to 10 mass %, and more preferably 0.5 to 5 mass % with respect to the total amount of the cosmetic.

### (C) Oil

The cosmetic according to the present invention includes (C) an oil. Examples of the component (C) include silicone oils, hydrocarbon oils, higher fatty acids, higher alcohols, ester oils, liquid oils, and oil-soluble ultraviolet absorbers. The component (C) is preferably selected from the group consisting of silicone oils, ester oils, hydrocarbon oils, and oil-soluble ultraviolet absorbers, and more preferably at least an oil-soluble ultraviolet absorber is selected therefrom. Here, ester oils do not include oil-soluble ultraviolet absorbers.

Examples of the silicone oils include chain polysiloxanes (such as dimethicone, diphenylsiloxy phenyl trimethicone, and diphenylpolysiloxane), cyclic polysiloxanes (such as octamethylcyclotetrasiloxane, decamethylcyclopentasiloxane, and dodecamethylcyclohexasiloxane), silicone resins forming a three-dimensional network structure, silicone rubber, various modified polysiloxanes (such as an amino-modified polysiloxane, a polyether-modified polysiloxane, an alkyl-modified polysiloxane, and a fluorine-modified polysiloxane), and acrylic silicones, and chain polysiloxanes are preferable.

Examples of the ester oils include octyl octanoate, nonyl nonanoate, cetyl octanoate, isopropyl myristate, octyldodecyl myristate, isopropyl palmitate, ethylhexyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, tripropylene glycol pivalate, PPG-3 dipivalate, diisostearyl malate, glyceryl di-2-heptylundecanoate, glyceryl diisostearate, trimethylolpropane tri-2-ethylhexanoate, trimethylolpropane triisostearate, pentaerythritol tetra-2-ethylhexanoate, glyceryl tri-2-ethylhexanoate (triethylhexanoin), glyceryl trioctanoate, glyceryl triisopalmitate, trimethylolpropane triisostearate, cetyl 2-ethylhexanoate-2-ethylhexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, phytosteryl/octyldodecyl lauroyl glutamate, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, cetyl ethylhexanoate, and phytosteryl macadamiate.

Examples of the hydrocarbon oils include isododecane, isohexadecane, isoparaffin, mineral oil (liquid paraffin), squalane, pristane, paraffin, squalene, and hydrogenated polydecene.

Examples of the higher fatty acids include lauric acid, myristic acid, palmitic acid, stearic acid, behenic acid, oleic acid, undecylenic acid, tall oil acid, isostearic acid, linolic acid, linoleic acid, eicosapentaenoic acid (EPA), and docosahexaenoic acid (DHA).

Examples of the higher alcohols include straight-chain alcohols (such as lauryl alcohol, cetyl alcohol, stearyl alcohol, behenyl alcohol, myristyl alcohol, oleyl alcohol, and cetostearyl alcohol) and branched alcohols (such as lanolin alcohol, cholesterol, phytosterol, hexyldodecanol, isostearyl alcohol, and octyldodecanol).

Examples of the liquid oils include avocado oil, camellia oil, macadamia nut oil, corn oil, olive oil, rapeseed oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, paulownia oil, Japanese tung oil, jojoba oil, germ oil, and triglycerin oil.

Examples of the oil-soluble ultraviolet absorbers include benzoic acid derivatives, salicylic acid derivatives, cinnamic acid derivatives, dibenzoylmethane derivatives, β,β-diphenylacrylate derivatives, benzophenone derivatives, phenylbenzimidazole derivatives, triazine derivatives, phenylbenzotriazole derivatives, anthranil derivatives, imidazoline derivatives, benzalmalonate derivatives, and 4,4-diarylbutadiene derivatives. It is generally known that if an ultraviolet absorber as an oil is blended in a cosmetic, the feel in use tends to deteriorate to cause, for example, a sticky feel. According to the present invention, a cosmetic can be provided that can realize a sufficient feel in use even if an oil-soluble ultraviolet absorber is included. Therefore, in a preferred embodiment of the present invention, the component (C) includes an oil-soluble ultraviolet absorber.

One or more kinds of the component (C) can be blended. The amount of the component (C) blended is preferably 20 to 70 mass %, and more preferably 30 to 60 mass % with respect to the total amount of the cosmetic.

### (D) Water

The cosmetic according to the present invention includes (D) water. As the water, water used in cosmetics, quasi-drugs, and the like can be used, and for example, purified water, ion-exchanged water, tap water, or the like can be used.

The amount of the water blended is preferably 10 to 60 mass %, and more preferably 20 to 50 mass % with respect to the total amount of the cosmetic according to the present invention.

### (E) Oil Phase Thickener

The cosmetic according to the present invention can further include (E) an oil phase thickener. The component (E) is a substance capable of adjusting the viscosity of the oil phase, and examples of the component (E) include dextrin fatty acid esters, sucrose fatty acid esters, and organo-modified clay minerals.

Dextrin fatty acid esters are an ester of dextrin or reduced dextrin and a higher fatty acid, and can be used without particular limitation as long as they are generally used in cosmetics. Dextrin or reduced dextrin is preferably used that has an average degree of saccharide polymerization of 3 to 100. As the constituent fatty acid of the dextrin fatty acid ester, a saturated fatty acid having 8 to 22 carbon atoms is preferably used. Specific examples of the dextrin fatty acid ester include dextrin palmitate, dextrin oleate, dextrin stearate, dextrin myristate, and dextrin palmitate/2-ethylhexanoate.

As the sucrose fatty acid ester, a saturated or unsaturated sucrose fatty acid ester can be preferably used that is an ester of a linear or branched fatty acid and has 12 to 22 carbon atoms. Specific examples of such a sucrose fatty acid ester include sucrose caprylate, sucrose caprate, sucrose laurate, sucrose myristate, sucrose palmitate, sucrose stearate, sucrose oleate, and sucrose erucate.

The organo-modified clay mineral is, for example, one kind of colloidal hydrous aluminum silicate having a three-layer structure, and examples of the organo-modified clay mineral include those obtained by modifying a clay mineral represented by the following formula with a quaternary ammonium salt cationic surfactant.

(X,Y)₂₋₃(Si,Al)₄O₁₀(OH)₂Z_{1/3}·nH₂O

In the formula,
X is Al, Fe(III), Mn(III), or Cr(III),
Y is Mg, Fe(II), Ni, Zn, or Li, and
Z is K, Na, or Ca.

Examples of such a quaternary ammonium salt cationic surfactant include dodecyltrimethylammonium chloride, myristyltrimethylammonium chloride, cetyltrimethylammonium chloride, stearyltrimethylammonium chloride, arachyltrimethylammonium chloride, behenyl trimethyl ammonium chloride, myristyldimethylethylammonium chloride, cetyldimethylethylammonium chloride, stearyldimethylethylammonium chloride, arachyldimethylethylammonium chloride, behenyldimethylethylammonium chloride, myristyldiethylmethylammonium chloride, cetyldiethylmethylammonium chloride, stearyldiethylmethylammonium chloride, arachyldiethylmethylammonium chloride, behenyldiethylmethylammonium chloride, benzyldimethylmyristylammonium chloride, benzyldimethylcetylammonium chloride, benzyldimethylstearylammonium chloride, benzyldimethylbehenylammonium chloride, benzylmethylethylcetylammonium chloride, benzylmethylethylstearylammonium chloride, dibehenyldihydroxyethylammonium chloride, corresponding bromides, and dipalmitylpropylethylammonium methylsulfate.

The organo-modified clay mineral is preferably selected from the group consisting of disteardimonium hectorite, quaternium-18 hectorite, stearalkonium hectorite, and distearyl dimethyl ammonium chloride-treated aluminum magnesium-silicate.

Examples of the component (E) other than those described above include ozokerite, ceresin, petrolatum, microcrystalline wax, cacao butter, coconut oil, hardened coconut oil, palm oil, palm kernel oil, hydrogenated palm oil, Japan wax kernel oil, hardened oil, Japan wax, hardened castor oil, shea butter, partially hydrogenated coconut oil, and partially hydrogenated jojoba oil.

One or more kinds of the component (E) can be blended. The amount of the component (E) blended is preferably 0.05 to 3 mass %, and more preferably 0.1 to 1 mass % with respect to the total amount of the cosmetic.

### (F) Nonionic Surfactant Having HLB of 1 to 7.5

The cosmetic according to the present invention can further include (F) a nonionic surfactant having an HLB of 1 to 7.5. The component (F) preferably has an HLB of 0.1 to 10, and more preferably 0.5 to 5. Here, in the present invention, the hydrophile-lipophile balance (HLB) of the component (F) can be determined with the Griffin method.

The component (F) is preferably selected from the group consisting of polyether-modified silicones, polyglycerin-modified silicones, polyglycerin fatty acid esters, and polyoxyalkylene fatty acid esters.

Examples of the polyether-modified silicones include PEG-9 polydimethylsiloxyethyl dimethicone, PEG-10 dimethicone, lauryl PEG-9 polydimethylsiloxyethyl dimethicone, and cetyl PEG/PPG-10/1 dimethicone.

Examples of the polyglycerin-modified silicones include bis-butyldimethicone polyglyceryl-3.

Examples of the polyglycerin fatty acid esters include polyglyceryl-2 diisostearate, polyglyceryl-6 triisostearate, polyglyceryl-10 pentastearate, polyglyceryl-10 pentaisostearate, polyglyceryl-10 pentaoleate, and polyglyceryl-6 polyricinoleate.

Examples of the polyoxyalkylene fatty acid esters include PEG-8 diisostearate, PEG-4 sorbitan triisostearate, and PEG-6 sorbitol hexastearate.

The component (F) is more preferably selected from the group consisting of PEG-9 polydimethylsiloxyethyl dimethicone, PEG-10 dimethicone, lauryl PEG-9 polydimethylsiloxyethyl dimethicone, cetyl PEG/PPG-10/1 dimethicone, bis-butyldimethicone polyglyceryl-3, polyglyceryl-2 diisostearate, polyglyceryl-6 polyricinoleate, and PEG-8 diisostearate.

One or more kinds of the component (F) can be blended. The amount of the component (F) blended is preferably 0.5 to 7 mass %, and more preferably 1 to 5 mass % with respect to the total amount of the cosmetic.

In the cosmetic in the present invention, an optional component usually used in cosmetics and pharmaceuticals can be blended in addition to the above-described components. Examples of the optional component include the following components, and one or more kinds of them can be blended as long as an effect of the present invention is exhibited.

Examples of the moisturizer include polyethylene glycol, propylene glycol, dipropylene glycol (DPG), glycerin, 1,3-butylene glycol (BG), erythritol, xylitol, sorbitol, maltitol, chondroitin sulfate, hyaluronic acid, mucoitinsulfuric acid, charonin acid, atelocollagen, cholesteryl-12-hydroxystearate, sodium lactate, dl-pyrrolidone carboxylates, and short-chain soluble collagen.

Examples of the lower alcohol include ethanol, 1-propanol, 2-propanol, isobutyl alcohol, and t-butyl alcohol.

Examples of the sequestering agent include 1-hydroxyethane-1,1-diphosphonic acid, 1-hydroxyethane-1,1-diphosphonic acid tetrasodium salt, edetate disodium, edetate trisodium, edetate tetrasodium, sodium citrate, sodium polyphosphate, sodium metaphosphate, gluconic acid, phosphoric acid, citric acid, succinic acid, edetic acid, and trisodium hydroxyethyl ethylenediamine triacetate.

Examples of the neutralizer include 2-amino-2-methyl-1-propanol, 2-amino-2-methyl-1,3-propanediol, potassium hydroxide, sodium hydroxide, triethanolamine, and sodium carbonate.

Examples of a pH adjuster include buffers such as lactic acid-sodium lactate, citric acid-sodium citrate, and succinic acid-sodium succinate.

Examples of the antioxidant include dibutylhydroxytoluene, butylhydroxyanisole, sodium pyrosulfite, and gallic acid esters.

Examples of the preservative include paraoxybenzoic acid esters such as methylparaben, ethylparaben, and butylparaben, benzoic acid, salicylic acid, sorbic acid, parachlorometacresol, hexachlorophene, benzalkonium chloride, chlorhexidine chloride, trichlorocarbanilide, photosensitizers, phenoxyethanol, and chlorphenesin.

Examples of the powder include color pigments such as titanium oxide, zinc oxide, cerium oxide, red iron oxide, yellow iron oxide, black iron oxide, ultramarine blue, talc, mica, sericite, kaolin, bentonite, clay, silicic acid, silicic anhydride, magnesium silicate, zinc stearate, fluorine-containing phlogopite, synthetic talc, barium sulfate, magnesium sulfate, calcium sulfate, boron nitride, bismuth oxychloride, alumina, zirconium oxide, magnesium oxide, chromium oxide, calamine, magnesium carbonate, and composites thereof, white pigments, cellulose, pearl agents, inorganic powders containing a body pigment, and organic resin powders, and these may be surface-treated.

In addition, drugs, aroma chemicals, surfactants other than those described above, and the like can also be appropriately blended.

Examples of the cosmetic according to the present invention include skin care cosmetics (such as lotions, milky lotions, creams, beauty essences, packs, and masks), makeup cosmetics (such as foundations and makeup bases), sunscreen cosmetics, and ointments. These embodiments are merely examples, and the cosmetic according to the present invention is not limited to these embodiments.

The method of emulsification for the cosmetic of the present invention is not particularly limited, and the cosmetic can be produced in accordance with a conventional method.

### EXAMPLES

The present invention will be described specifically with reference to the following Examples, but the present invention is not limited to these Examples. The content is shown in mass % with respect to the total amount unless otherwise specified.

### Examples 1 and 2 and Comparative Examples 1 to 7

Cosmetics of Examples 1 and 2 and Comparative Examples 1 to 7 were prepared at combinations shown in Tables 1 and 2. The numerical values of the components in the tables are shown in mass %.

TABLE 1

| | Example | | Comparative Example | | | | | | |
|---|---|---|---|---|---|---|---|---|---|
| | 1 | 2 | 1 | 2 | 3 | 4 | 5 | 6 | 7 |
| 1-(2-Hydroxyethyl)-2- | 3 | 1 | — | — | 3 | 3 | — | — | — |
| imidazolidinone | | | | | | | | | |
| Terephthalylidene | 3.4 | 3.4 | — | — | — | — | 3.4 | — | 3.4 |
| dicamphor sulfonic acid | | | | | | | | | |
| Phenylbenzimidazole | — | — | — | 3 | — | 3 | — | — | — |
| sulfonic acid | | | | | | | | | |
| Triethanolamine | — | — | — | 2.2 | — | 2.2 | — | — | — |
| Glycerin | — | — | — | — | — | — | — | 3 | 3 |
| Isododecane | 47.5 | 47.5 | 47.5 | 47.5 | 47.5 | 47.5 | 47.5 | 47.5 | 47.5 |
| Water | Balance | Balance | Balance | Balance | Balance | Balance | Balance | Balance | Balance |
| PEG-10 dimethicone | 3 | 3 | 3 | 3 | 3 | 3 | 3 | 3 | 3 |
| Disteardimonium hectorite | 0.2 | 0.2 | 0.2 | 0.2 | 0.2 | 0.2 | 0.2 | 0.2 | 0.2 |
| Dextrin palmitate-treated | 10 | 10 | 10 | 10 | 10 | 10 | 10 | 10 | 10 |
| zinc oxide | | | | | | | | | |
| Ethanol | 5 | 5 | 5 | 5 | 5 | 5 | 5 | 5 | 5 |
| Phenoxyethanol | 0.5 | 0.5 | 0.5 | 0.5 | 0.5 | 0.5 | 0.5 | 0.5 | 0.5 |
| Total | 100 | 100 | 100 | 100 | 100 | 100 | 100 | 100 | 100 |
| Oily feel | A | A | C | C | C | C | C | C | C |
| Smoothness | A | B | C | C | A | A | C | C | C |
| Freshness | A | A | C | C | C | C | A | C | A |

The cosmetic prepared above was applied to the skin of 10 expert panelists, and the panelists evaluated the “oily feel”, the “smoothness”, and the “freshness” during the application. Determination was made in accordance with the following criteria on the basis of the evaluation of each expert panelist. The obtained results are as shown in Table 1.

### [Oily Feel]

A: Seven or more of the 10 panelists answered that an oily feel was absent during application.

B: Four or more and 6 or less of the 10 panelists answered that an oily feel was absent.

C: Three or less of the 10 panelists answered that an oily feel was absent.

### [Smoothness]

A: Seven or more of the 10 panelists answered that smoothness was present during application.

B: Four or more and 6 or less of the 10 panelists answered that smoothness was present.

C: Three or less of the 10 panelists answered that smoothness was present.

### [Freshness]

A: Seven or more of the 10 panelists answered that freshness was present during application.

B: Four or more and 6 or less of the 10 panelists answered that freshness was present.

C: Three or less of the 10 panelists answered that freshness was present.

1. A water-in-oil emulsion cosmetic comprising:
(A) a cyclic carboxamide derivative or a salt of the cyclic carboxamide derivative, the cyclic carboxamide derivative represented by Formula (1): 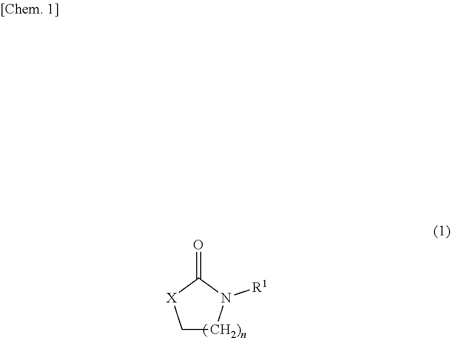
wherein
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3;
(B) a water-soluble benzylidene camphor derivative or a benzylidene camphor derivative salt;
(C) an oil; and
(D) water.

2. The cosmetic according to claim 1,
wherein in Formula (1) of the component (A),
R¹ represents a hydroxyalkyl group having 1 to 3 carbon atoms,
X represents —CH₂— or —NH—, and
n is 1.

3. The cosmetic according to claim 1, wherein the component (A) is 1-(2-hydroxyethyl)-2-imidazolidinone.

4. The cosmetic according to claim 1, wherein an amount of the component (A) blended is 0.05 to 5 mass % with respect to a total amount of the cosmetic.

5. The cosmetic according to claim 1, wherein the component (B) is selected from the group consisting of terephthalylidene dicamphor sulfonic acid, benzylidene camphor sulfonic acid, and camphor benzalkonium methosulfate.

6. The cosmetic according to claim 1, wherein an amount of the component (B) blended is 0.05 to 10 mass % with respect to the total amount of the cosmetic.
